(19) United States
(12) Patent Application Publication
(10) Pub. No.: US 2021/0299337 A1
(43) Pub. Date: Sep. 30, 2021

(54) COMPACT CATHETER DRAINAGE BAG ASSEMBLY

(71) Applicant: Melchizedek Dontell Scott, McDonough, GA (US)

(72) Inventor: Melchizedek Dontell Scott, McDonough, GA (US)

(21) Appl. No.: 16/829,588

(22) Filed: Mar. 25, 2020

(51) Int. Cl. A61M 1/00; A61J 1/14; A61J 1/10

(52) U.S. Cl. CPC A61M 1/0019; A61M 1/0088; A61M 2202/0496; A61J 1/10; A61M 2205/273; A61J 1/1412

(57) ABSTRACT

A catheter drainage bag assembly, a urine-collecting device, and a disposable, portable urine drainage kit is provided. The assembly includes a tube, a drainage bag, and a connector. The tube is secured to a mouth of the drainage bag by way of the connector. The urine-collecting device includes a foldable waterproof pouch with an inlet, a tubular reinforcement; and a connector connecting the inlet of the foldable waterproof pouch to the tubular reinforcement. The disposable, portable urine drainage kit includes at least one tube, at least one connector, and at least one urine drainage bag with a mouth that secures to the tube with the connector.

### BACKGROUND OF THE INVENTION

The present invention relates to catheter drainage bags and, more particularly, to compact, disposable drainage bags. An individual confined to a wheelchair often finds that his or her activities are restricted for several reasons. One reason is that suitable facilities must be available for catheterization. Existing products are generally big and bulky, making such devices awkward to transport, to use and to store. Moreover, the existing products require the catheter to be affixed to the drainage bag. In addition, the existing devices are not designed to be disposable with collected urine.

As can be seen, there is a need for a discreet drainage bag assembly that may be used anywhere and allows easy disposal of urine even when appropriate facilities are not readily available. The present invention makes it easy for an individual to catheterize anywhere into a compact bag and throw it away. The inventive assembly is small and compact and is therefore easily and discreetly transported. The small, compact, and disposable nature of the inventive bag assembly provides the user more freedom, allowing the user to travel to previously inaccessible locations and simply “catch and go”.

### SUMMARY OF THE INVENTION

In one aspect of the present invention, a catheter drainage bag assembly is provided comprising a tube, a drainage bag, and a connector. The tube is secured to a mouth of the drainage bag by way of the connector.

In another aspect of the present invention, a urine-collecting device is provided comprising a foldable waterproof pouch with an inlet, a tubular reinforcement; and a connector connecting the inlet of the foldable waterproof pouch to the tubular reinforcement.

In yet another aspect of the present invention, a disposable, portable urine drainage kit, is provided. The kit includes at least one tube, at least one connector, and at least one urine drainage bag with a mouth that secures to the tube with the connector.

These and other features, aspects and advantages of the present invention will become better understood with reference to the following drawings, description and claims.

### BRIEF DESCRIPTION OF THE DRAWINGS

FIG. 1 is a top front perspective view of an embodiment of the inventive catheter bag in an open position;

FIG. 2 is a front elevational view of the catheter bag of FIG. 1;

FIG. 3 is a rear elevational view of the catheter bag of FIG. 1;

FIG. 4 is a right elevational view of the catheter bag of FIG. 1;

FIG. 5 is a left elevational view of the catheter bag of FIG. 1;

FIG. 6 is a plan view of the catheter bag of FIG. 1;

FIG. 7 is a bottom elevational view of the catheter bag of FIG. 1;

FIG. 8 is a top front perspective view of an embodiment of the inventive catheter bag in a stored position;

FIG. 9 is a front elevational view of the catheter bag of FIG. 8;

FIG. 10 is a rear elevational view of the catheter bag of FIG. 8;

FIG. 11 is a right elevational view of the catheter bag of FIG. 8;

FIG. 12 is a left elevational view of the catheter bag of FIG. 8;

FIG. 13 is a plan view of the catheter bag of FIG. 8;

FIG. 14 is a bottom elevational view of the catheter bag of FIG. 8; and

FIG. 15 is an exploded view of the catheter bag of FIG. 1.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is an assembly comprising a urine drainage bag to receive urine, a tube to keep the bag open and to allow passage of the catheter, and a connector to secure the bag to the tube.

The connector of the invention includes, but is not limited to, an element selected from the group consisting of a glue, a tape, string, rope, one or more elastic bands, one or more clamps, one or more zip ties, and a combination thereof. The types of glue and tape are not limited and may include any type of adhesive tape such as packing tape, duct tape, and electrical tape. The types of glue are also not limited and may include cyanoacrylate adhesive, hot glue, spray adhesive, epoxy, polyurethane, and glue stick. In some cases, the bag may be welded onto the tube. In some embodiments, the invention further comprises an elastic band, such as a rubber band, that serves to secure the device in stored position. The elastic band may also serve as a closure of the bag for disposal.

The tube may be manufactured of any suitable material. For example, the material may be selected from the group consisting of silicone, cardboard, rubber, metal, bamboo, rigid or pliable plastic, a wide straw, and a combination thereof. The size of the tube may vary, in part, due to the gender of the user. The tube may have a diameter in the range between about ¼ inch and about 2 inches, preferably between about ½ inch to about 1 inch. The length of the tube is not particularly limited.

The bag is generally manufactured from a material that retains water without bursting or leaking, for example from a seam, and is foldable and/or collapsible, making the bag easy to store within the tube prior to use. For example, the bag may be manufactured from polymers including ethylene-vinylacetate copolymer (EVA), polyvinyl chloride (PVC), polyethylene (PE), and polypropylene (PP).

In some embodiments, the tube may be removed from the bag and reused, if desired. The bag may be securely closed, for example by tying, and discarded.

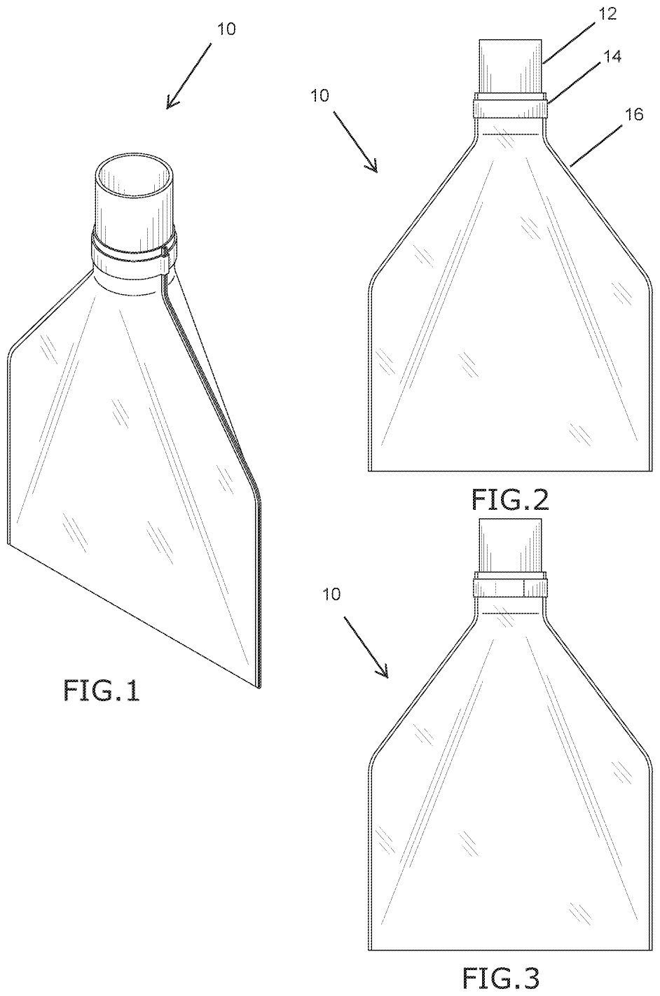

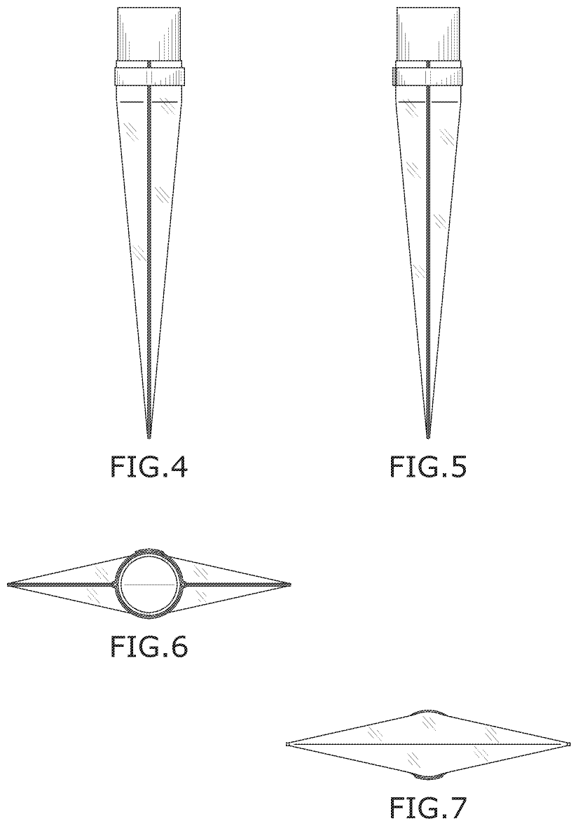

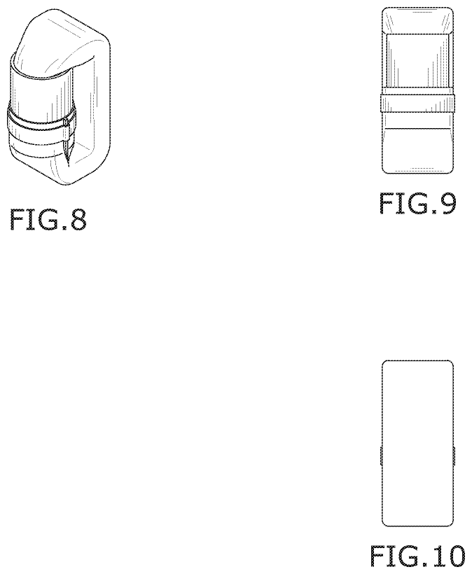

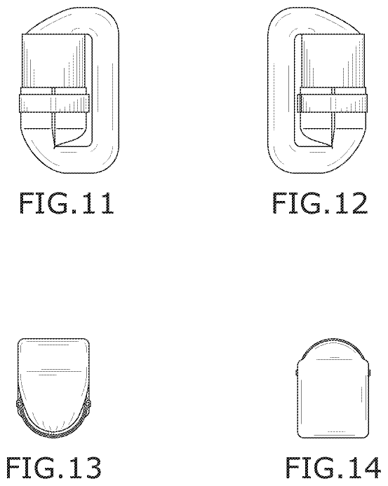

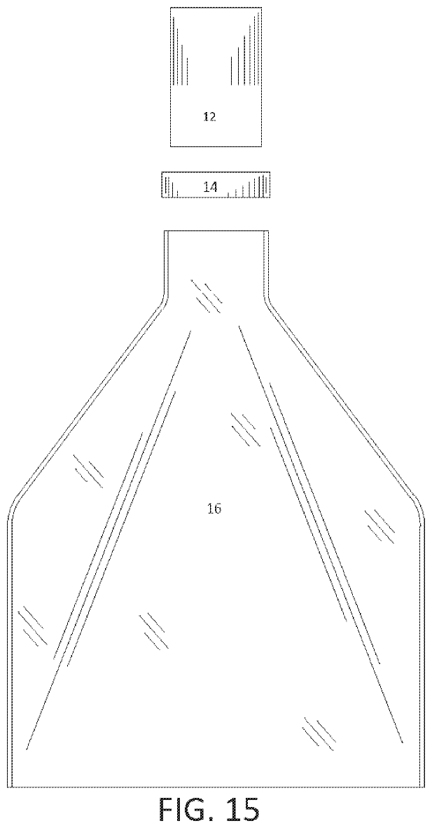

Referring to FIGS. 1-15, FIGS. 1-7 illustrate an embodiment of the catheter bag assembly 10 in an open position. The tube 12 may be secured to the drainage bag 16 with a connector 14. FIGS. 8-14 illustrate the catheter bag assembly in a stored position, with the drainage bag folded into the tube. FIG. 15 is an exploded view of the catheter bag of FIG. 1, showing the tube 12, connector 14, and bag 16 components. The mouth or inlet 18 of the bag 16 is also illustrated.

The catheter bag assembly 10 of the present invention may be provided as an assembled device or as a kit comprising a tube 12, one or more connectors 14, and one or more urine drainage bags 16. With respect to a kit embodiment, the tube 12 may be inserted into the bag 16 and the connector 14 may be secured around the bag. Alternatively, the bag 16 may be inserted into the tube 12 and an inlet or mouth 18 of the bag 16 may be folded around the tube 12 prior to securing the bag 16 to the tube 12 with the connector 14.

A method of using the present invention may include the following. The catheter bag assembly 10 disclosed above may be provided as an assembled device or as a kit. A user may secure the bag 16 to the tube 12, and then an outlet of a catheter (not shown) may be inserted through the tube 12 into the bag 16 so that the user may release urine to drain into the bag 16.

Once the user is finished releasing urine, the catheter bag assembly 10 may be disposed in a suitable receptacle. In some cases, the user may drain the bag 16 contents prior to disposing of the catheter bag assembly 10. In some embodiments, the connector 14 may be removed and the bag 16 may be withdrawn from the tube 12. The bag 16 may be tied closed, sealed, or secured with a closure (not shown) and disposed. The tube 12 may be retained for reuse.

It should be understood, of course, that the foregoing relates to exemplary embodiments of the invention and that modifications may be made without departing from the spirit and scope of the invention as set forth in the following claims.

What is claimed is:

1. A catheter drainage bag assembly comprising:
a. a tube;
b. a drainage bag; and
c. a connector;
wherein the tube is secured to a mouth of the drainage bag by way of the connector.

2. The catheter drainage bag assembly of claim 1, wherein the drainage bag is foldable and is configured to be folded into the tube for storage prior to use.

3. The catheter drainage bag assembly of claim 1, wherein the tube keeps the mouth of the drainage bag open such that a catheter may be inserted into the drainage bag.

4. The catheter drainage bag assembly of claim 1, wherein the connector and the drainage bag are removable for disposal.

5. The catheter drainage bag assembly of claim 1, wherein the drainage bag is sealable or is further provided with a closure to retain urine for disposal.

6. The catheter drainage bag assembly of claim 2, wherein the tube keeps the mouth of the drainage bag open such that a catheter may be inserted into the drainage bag; the connector and the drainage bag are removable for disposal; and the drainage bag is sealable or is further provided with a closure to retain urine for disposal.

7. A urine-collecting device comprising:
a. a foldable waterproof pouch with an inlet;
b. a tubular reinforcement; and
c. a connector connecting the inlet of the foldable waterproof pouch to the tubular reinforcement.

8. The urine-collecting device of claim 7, wherein the foldable waterproof pouch with the inlet is configured to fold into the tubular reinforcement for storage.

9. A disposable, portable urine drainage kit, comprising:
a. at least one tube;
b. at least one connector; and
c. at least one urine drainage bag;
wherein the urine drainage bag has a mouth that secures to the tube with the connector.

10. The disposable, portable urine drainage kit of claim 9, wherein the mouth of the urine drainage bag has a diameter and a length configured to insert into the tube and to extend over an outer circumference of the tube, and wherein the connector is configured to secure the mouth of the urine drainage bag to the tube.

11. The disposable, portable urine drainage kit of claim 9, wherein the mouth of the urine drainage bag has a diameter and a length configured to surround an outer circumference of the tube, and wherein the connector is configured to secure the mouth of the urine drainage bag to the tube.
